(19) United States
(12) Patent Application Publication
(10) Pub. No.: US 2025/0364092 A1
(43) Pub. Date: Nov. 27, 2025

(54) Material Search Method, Material Search System, Program, And Recording Medium

(71) Applicant: SEMICONDUCTOR ENERGY LABORATORY CO., LTD., Kanagawa-ken (JP)

(72) Inventors: Kazuki TANEMURA, Isehara, Kanagawa (JP); Mayumi MIKAMI, Atsugi, Kanagawa (JP); Yohei MOMMA, Isehara, Kanagawa (JP); Kazuki HIGASHI, Atsugi, Kanagawa (JP)

(73) Assignee: SEMICONDUCTOR ENERGY LABORATORY CO., LTD., Kanagawa-ken (JP)

(21) Appl. No.: 18/873,846

(22) PCT Filed: Jun. 12, 2023
(86) PCT No.: PCT/IB2023/056014

§ 371 Date: Dec. 11, 2024

(30) Foreign Application Priority Data
Jun. 24, 2022 (JP) 2022-101884
Jul. 15, 2022 (JP) 2022-114169

(51) Int. Cl. G16C 60/00; G06F 16/908; G16C 20/20; G16C 20/90

(52) U.S. Cl. CPC G16C 60/00; G06F 16/908; G16C 20/20; G16C 20/90

(57) ABSTRACT

A novel material search method and a novel material search system are provided. P peak positions are obtained in descending order of peak intensity from a plurality of peaks appearing in an input XRD profile of a sample. In addition, for each of a plurality of pieces of physical property data of known materials registered in a material database, a record including R peak positions is generated in descending order of peak intensity. From a plurality of records, a record including peaks matching or substantially matching all of the P peak positions of the sample is searched for. In the case where the corresponding record is found, it is determined that the sample matches the known material registered in the material database. Furthermore, at least part of information related to the known material is output. R is preferably greater than P, and R is preferably less than or equal to 6 times P.

### TECHNICAL FIELD

One embodiment of the present invention relates to a material search method, a material search system, a program, and a recording medium.

Note that one embodiment of the present invention is not limited to the above technical field. The technical field of the invention disclosed in this specification and the like relates to an object, a method, or a manufacturing method. One embodiment of the present invention relates to a process, a machine, manufacture, or a composition of matter.

### BACKGROUND ART

In recent years, various materials such as an inorganic material and an organic material have been actively developed in various technical fields. As a method for evaluating material's characteristics, an analysis method using an X-ray diffraction (XRD) method (also referred to as “XRD analysis”) is known. Examples of the XRD analysis include a powder X-ray diffraction method, and the XRD analysis can nondestructively evaluate crystallinity and orientation, or identify or estimate a material, for example. In particular, a powder X-ray diffraction method is widely used as a method for analyzing a polycrystal.

In the XRD analysis, a sample is irradiated with a constant-wavelength X-ray by changing incident angles, and the intensity of the reflected X-ray is measured to obtain a diffraction pattern inherent in the substance of the sample (also referred to as “2θ/θ measurement”, “2θ/ω measurement”, or “Out-of-plane measurement”). From the obtained diffraction pattern (also referred to as “XRD profile”, “XRD spectrum”, or “powder pattern”), elements constituting the sample, crystallinity, orientation, and the like can be known.

In addition, the XRD analysis is one of methods used for analysis of the crystal structure of a positive electrode active material. XRD data can be analyzed with the use of crystal structure data stored in the ICSD (Inorganic Crystal Structure Database) introduced in Non-Patent Document 1. For example, the ICSD can be referred to for the lattice constant of the lithium cobalt oxide described in Non-Patent Document 2.

On the other hand, in the XRD analysis, advanced specialized knowledge of crystallography or the like is required for analyzing diffraction patterns and identifying materials, and it takes time for analysis.

As examples of identification of materials using diffraction patterns, a method in which a corresponding substance is searched for with a database (e.g., Hanawalt Index) from the peak positions of the three most intense lines (indicated by interplanar spacings in some cases), a method in which an error window is set around the peak positions and relative intensities and matching or mismatching is determined depending on whether the peak positions and relative intensities in the ICDD-PDF file are within the error window, a method in which determination is made based on a probability theory (SANDMAN (Search and Match on Nova)), and the like are proposed.

### REFERENCES

### Non-Patent Documents

[Non-Patent Document 1] Belsky, A. et al., “New developments in the Inorganic Crystal Structure Database (ICSD): accessibility in support of materials research and design”, Acta Cryst., (2002) B58 364-369.
[Non-Patent Document 2] Akimoto, J.; Gotoh, Y.; Oosawa, Y. “Synthesis and structure refinement of LiCoO₂ single crystals”, Journal of Solid State Chemistry (1998) 141, p. 298-302.

### SUMMARY OF THE INVENTION

### Problems to be Solved by the Invention

However, a large amount of data of a variety of technical fields is stored in a general XRD database and analysis software, and there is a problem in that the number of candidate substances (also referred to as “candidate materials”) to be selected is too large in a search method using these data. In addition, software for analyzing the diffraction patterns is incorporated in an XRD analysis apparatus in many cases, and thus the opportunity for the user to use the software is limited. Furthermore, although the composition and the like can be narrowed down, there is a problem in that it is not always possible to search for substances from a database of a technical field in accordance with the purpose.

For example, the XRD analysis is also widely used in the field of lithium-ion batteries in synthesis of materials, deterioration analysis, and the like. However, data analysis is difficult for engineers who are not necessarily skilled in crystallography.

In particular, in the case of a lithium-ion battery, an electrode in which an active material, a conductive additive, and a binder are mixed, applied to a current collector, and then pressed (also referred to as “electrode material mixture”) is used. In the evaluation of the electrode material mixture, analysis is more difficult because a plurality of materials are mixed and the active material is aligned due to pressing for increasing the density of the electrode, for example.

Thus, it is required that a user can construct a database used for a search and perform analysis easily with a given computer without the use of the software incorporated in the XRD analysis apparatus. Furthermore, it is not preferable for an unskilled user that a large number of candidate materials are displayed after the search; thus, only one candidate material is desired to be shown. When the candidate material is selected automatically, the existence of an impurity phase might hinder an accurate selection; thus, the database of materials is desirably searched by the peak position and the range of the peak.

One object of one embodiment of the present invention is to provide a material search system capable of searching for materials without advanced specialized knowledge. One object of one embodiment of the present invention is to provide a material search method capable of searching for materials without advance specialized knowledge. One object of one embodiment of the present invention is to provide a material search system in which cost is reduced. One object of one embodiment of the present invention is to provide a material search method in which cost is reduced. One object of one embodiment of the present invention is to provide a novel material search method. One object of one embodiment of the present invention is to provide a novel material search system.

Note that the description of these objects does not preclude the existence of other objects. One embodiment of the present invention does not need to achieve all these objects. Note that other objects will be apparent from the description of the specification, the drawings, the claims, and the like, and other objects can be derived from the description of the specification, the drawings, the claims, and the like.

### Means for Solving the Problems

One embodiment of the present invention is a material search method and a material search system that achieve a low-cost and accurate material (substance) search using an XRD profile obtained by 2θ/θ measurement, which is a general method for an XRD analysis.

Here, the 2θ/θ measurement for the XRD analysis is described. The 2θ/θ measurement is a method in which an X-ray is incident on a sample at an angle θ with respect to the horizontal direction and the intensity of the reflected X-ray is detected at an angle 2θ with respect to the horizontal direction. The intensity of the reflected X-ray is detected at a position 2θ while θ, which is the incident angle, is changed. When the XRD profile indicating the intensity of the reflected X-ray with respect to the change in the 2θ value is analyzed, evaluation of crystallinity and orientation, estimation of materials, and the like can be realized. For example, a constituent material of a sample can be estimated from the peak positions and intensities appearing in the XRD profile. Estimating constituent materials of a sample is sometimes referred to as identification. Note that the XRD analysis is not limited to the 2θ/θ measurement. For example, there is also 2θ measurement. The 2θ measurement is a method in which the incident angle of an X-ray with which a sample is irradiated is fixed and the position of a detector is changed, whereby the intensity of the reflected X-ray is measured. Since θ and 2θ are angles, the unit of θ and 2θ is sometimes indicated by “degree”, “deg.”, or “°” in this specification and the like.

In the analysis of a sample in a polycrystalline state (e.g., a solid or powdered sample), a plurality of peaks inherent in a substance appear in the XRD profile. A constituent material of a crystalline sample can be estimated with the use of the peak positions and intensities appearing in the XRD profile and a material database including physical property data of known materials. A user may freely add or delete data to or from the material database. When materials that are not handled by the user or are assumed to be definitely not included are excluded from the material database, estimation can be performed more accurately.

Specifically, the peak positions and intensities appearing in the XRD profile of the sample are used as clues for searching in the material database. When searched for in the material database, a matching or substantially matching material is difficult to determine with the use of just one peak among the plurality of peaks appearing in the XRD profile. Thus, the materials are searched for with the use of the plurality of peaks appearing in the XRD profile. The plurality of peaks used for searching for the materials are determined in descending order of peak intensity. Note that in this specification and the like, “peak intensity” refers to the height of a peak.

In one embodiment of the present invention, for example, materials included in a sample are searched for using three peaks in descending order of peak intensity. Specifically, the peak having the highest peak intensity is referred to as a first peak, the peak having the second highest peak intensity is referred to as a second peak, and the peak having the third highest peak intensity is referred to as a third peak, and the positions at which the first to third peaks appear are compared with the peak positions of known materials registered in the material database, whereby the presence or absence of physical property data matching or substantially matching the sample is determined. Note that the position of the peak appearing in the XRD profile is defined by a value of 2θ.

An error is generated in the intensity of each of the plurality of peaks appearing in the XRD profile due to the conditions of the sample, the installation condition of the sample, or the like, and the order of peak intensity is changed in some cases. For example, a peak that should be determined as the second peak is determined as the third peak in some cases. Thus, when the order of peak intensity is strictly used for the determination of materials matching or substantially matching the sample, it becomes difficult to accurately detect the materials. Thus, a large number of candidate search results are presented in the conventional Hanawalt method or the like in some cases. As a result, an unfamiliar worker cannot determine which is the correct search result in some cases.

For example, in the case where LiCoO₂, which is generally used as a positive electrode active material, is an unaligned LiCoO₂ powder, the first peak is a reflection from a (003) plane and the second peak is a reflection from a (104) plane; meanwhile, in the case where LiCoO₂ is oriented to the (001) plane, the reflection from the (003) plane of the first peak becomes significantly stronger than the reflections from the other planes, and the second peak becomes a reflection from a (006) plane in some cases. In such a case, when comparison is made with the intensity ratio in the material database, a search cannot be performed correctly in some cases.

For example, a material search for a sample can be performed by the following method. First, the peak positions of P peaks are obtained in descending order of peak intensity from the plurality of peaks appearing in the input XRD profile of the sample. In addition, for each of the plurality of pieces of physical property data of known materials registered in the material database, a record including the peak positions of R peaks is generated in descending order of peak intensity. When the record including peaks matching or substantially matching all of the P peak positions of the sample is searched for from a data set including the plurality of records, and in the case where the corresponding record is found, it is determined that the sample matches a known material related to the record. After that, at least part or the whole of the physical property data of the known material that is determined to match the sample may be output to a display device or the outside. Note that P is an integer greater than or equal to 2 and less than or equal to 10, and R is an integer greater than P.

Note that in the case where a plurality of matching candidate materials are presented, a processing of searching again with a smaller value of a relative difference E (detection range) used for determining whether peak positions match may be repeated until the searched candidates are narrowed down to one. Alternatively, in the case where no matching candidate materials is displayed, the processing of searching again with a greater value of the relative difference E may be repeated until one searched candidate is found.

In the case where a determination of match is made but a relatively strong peak that is not used for determination is observed in the XRD profile, for example, another candidate material may be directly searched with the use of information on the peak position, the peak intensity, the reflective surface, and the like in the material database.

Another embodiment of the present invention is a material search method using an XRD profile and a first data set. The first data set includes a plurality of records each including R first peak positions extracted from each of a plurality of pieces of physical property data of known materials in descending order of peak intensity. A plurality of second peak positions and intensities are identified from the XRD profile of a first material. P second peak positions are obtained in descending order of peak intensity from the plurality of second peak positions and intensities. In the first data set, the record including the first peak positions matching the P second peak positions is searched for. In the case where a matched record is found, the first material is determined to be the same as the known material related to the record. P is an integer greater than or equal to 2 and less than or equal to 10. R is an integer greater than P.

Another embodiment of the present invention is a material search system using an XRD profile and a first data set. The first data set includes a plurality of records each including R first peak positions extracted from each of a plurality of pieces of physical property data of known materials in descending order of peak intensity. The material search system has a function of identifying a plurality of second peak positions and intensities from the XRD profile of a first material. The material search system has a function of obtaining P second peak positions in descending order of peak intensity from the plurality of second peak positions and intensities. In the first data set, the material search system has a function of searching for the record including the first peak position matching each of the P second peak positions. In the case where a matched record is found, the material search system has a function of determining the first material to be the same as the known material related to the record. P is an integer greater than or equal to 2 and less than or equal to 10. R is an integer greater than P.

R is preferably less than or equal to 6 times P, further preferably less than or equal to 3 times P.

The record may include R plane indices corresponding to the R first peak positions. The material search system may have a function of calculating a lattice constant of the first material determined to be the same as the known material using the first peak positions and the plane indices.

Another embodiment of the present invention is a material search system using an XRD profile and a second data set. The second data set includes a plurality of records. The record includes a name of a material having a peak at a peak position in a specified range and the peak position. The material search system has a function of displaying the XRD profile on a display device, a function of displaying the second data set on the display device, and a function of displaying, on the display device, a vertical marker indicating the peak position of the record selected from the second data set. The peak included in the record preferably has a relative intensity in the specified range. The vertical marker may be superimposed on the XRD profile.

Another embodiment of the present invention is a program for executing the above-described material search method on a computer. Another embodiment of the present invention is a program for implementing the above-described material search system with a computer. Another embodiment of the present invention is a computer-readable recording medium that stores the program.

### Effect of the Invention

According to one embodiment of the present invention, a material search system capable of searching for materials without specialized advanced knowledge can be provided. According to one embodiment of the present invention, a material search method capable of searching for materials without specialized advanced knowledge can be provided. According to one embodiment of the present invention, a material search system in which cost is reduced can be provided. According to one embodiment of the present invention, a material search method in which cost is reduced can be provided. According to one embodiment of the present invention, a novel material search method can be provided. According to one embodiment of the present invention, a novel material search system can be provided.

Note that the description of these effects does not preclude the existence of other effects. Note that one embodiment of the present invention does not need to have all of these effects. Other effects will be apparent from the descriptions of the specification, the drawings, the claims, and the like, and other effects can be derived from the descriptions of the specification, the drawings, the claims, and the like.

### BRIEF DESCRIPTION OF THE DRAWINGS

FIG. 1A is a block diagram illustrating a structure example of a material search system of one embodiment of the present invention. FIG. 1B is a graph showing an example of an XRD profile.

FIG. 2 is a table showing an example of a material database and physical property data.

FIG. 3 is a table showing an example of a data set and a record.

FIG. 4 is a flow chart for illustrating an operation example of a material search system.

FIG. 5A is a graph showing an example of an XRD profile. FIG. 5B is a table showing peak positions of first to third peaks and relative values of the peak intensities.

FIG. 6 is a table comparing peak positions of a sample with peak positions registered in a record.

FIG. 7 is a diagram illustrating an example of a search result displayed on a display screen of a display device.

FIG. 8A and FIG. 8B are flow charts for illustrating an operation example of a material search system.

FIG. 9A and FIG. 9B are flow charts for illustrating an operation example of a material search system.

FIG. 10A is a diagram illustrating an example of a search result displayed on the display device.

FIG. 10B is a diagram illustrating an example of a filter condition. FIG. 10C is a table showing an example of a data set.

FIG. 11A is a diagram illustrating an example of a filter condition. FIG. 11B is a table showing an example of a data set. FIG. 11C is a graph showing an example of a display screen.

FIG. 12A to FIG. 12C are diagrams each showing a filter condition.

### MODE FOR CARRYING OUT THE INVENTION

Embodiments will be described in detail with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments. Note that in structures of the invention described below, the same portions or portions having similar functions are denoted by the same reference numerals in different drawings, and description thereof is not repeated.

Ordinal numbers such as “first” and “second” in this specification and the like are used in order to avoid confusion among components and do not denote the priority or the order such as the order of steps or the stacking order. A term without an ordinal number in this specification and the like may be provided with an ordinal number in the in order to avoid confusion among components. Furthermore, a term with an ordinal number in this specification and the like may be provided with a different ordinal number in the SCOPE OF CLAIMS. Furthermore, even when a term is provided with an ordinal number in this specification and the like, the ordinal number might be omitted in the SCOPE OF CLAIMS and the like.

In this specification and the like, a space group is represented using the short notation of the international notation (or the Hermann-Mauguin notation). In addition, the Miller index is used for the expression of crystal planes and crystal orientations. In the crystallography, a bar is placed over a number in the expression of space groups, crystal planes, and crystal orientations; in this specification and the like, because of format limitations, space groups, crystal planes, and crystal orientations are sometimes expressed by placing “−” (a minus sign) in front of the number instead of placing a bar over the number. Furthermore, an individual direction which shows an orientation in a crystal is denoted with “[]”, a set direction which shows all of the equivalent orientations is denoted with “<>”, an individual plane which shows a crystal plane is denoted with “( )”, and a set plane having equivalent symmetry is denoted with “{}”. A trigonal system represented by the space group R-3m is generally represented by a composite hexagonal lattice for easy understanding of the structure and is also represented by a composite hexagonal lattice in this specification and the like unless otherwise specified. In some cases, not only (hkl) but also (hkil) is used as the Miller index. Here, i is −(h+k). In this specification and the like, a crystal plane or the like in the space group R-3m is represented with use of a composite hexagonal lattice, unless otherwise specified.

### Embodiment 1

In this embodiment, a structure example and an operation example of a material search system 100 of one embodiment of the present invention will be described.

### <Structure Example of Material Search System>

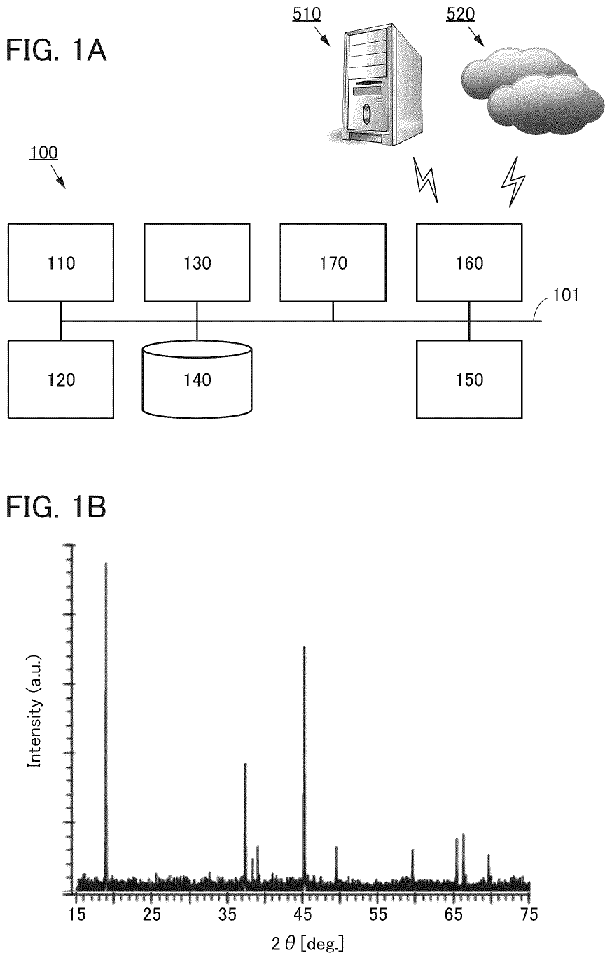

FIG. 1A is a block diagram illustrating a structure example of the material search system 100 of one embodiment of the present invention. The material search system 100 includes a control device 110, an arithmetic device 120, a memory device 130, an auxiliary memory device 140, an input/output device 150, a communication device 160, and a display device 170. The devices are electrically connected to each other through a bus line 101.

### [Control Device 110 and Arithmetic Device 120]

The control device 110 has a function of controlling the operation of the devices included in the material search system 100. The arithmetic device 120 has a function of executing arithmetic processing related to the material search. A central processing unit (CPU) or the like can be used as the control device 110, for example. As the arithmetic device 120, a CPU, a GPU (Graphics Processing Unit), or the like can be used, for example.

The control device 110 and the arithmetic device 120 may be achieved using a PLD (Programmable Logic Device) such as an FPGA (Field Programmable Gate Array) or an FPAA

### (Field Programmable Analog Array).

An arithmetic result obtained in the arithmetic device 120 is displayed on the display device 170. The arithmetic result obtained in the arithmetic device 120 is stored in the memory device 130 or the auxiliary memory device 140. The arithmetic result obtained in the arithmetic device 120 is output to the outside through the input/output device 150 or the communication device 160.

### [Memory Device 130]

The memory device 130 is preferably a memory which has a function of storing programs and parameters related to the operation of the material search system 100 and at least part of which is rewritable. For example, the memory device 130 can include a volatile memory such as a RAM (Random Access Memory) or a nonvolatile memory such as a ROM (Read Only Memory).

For example, a DRAM (Dynamic Random Access Memory) is used as a RAM provided in the memory device 130. A memory space as a workspace of the material search system 100 is assigned to part of the RAM. An operating system, an application program, data, and the like that are stored in the auxiliary memory device 140 are read into the RAM for execution.

As the ROM provided in the memory device 130, a mask ROM, an OTPROM (One Time Programmable Read Only Memory), an EPROM (Erasable Programmable Read Only Memory), or the like can be used. Examples of the EPROM include a UV-EPROM (Ultra-Violet Erasable Programmable Read Only Memory) which can erase stored data by ultraviolet irradiation, an EEPROM (Electrically Erasable Programmable Read Only Memory), and a flash memory. In the ROM, a BIOS (Basic Input/Output System), a firmware, and the like for which rewriting is not needed can be stored.

### [Auxiliary Memory Device 140]

The auxiliary memory device 140 is a memory device that stores an operating system, an application program, data, and the like. In addition, a variety of parameters that are used in the arithmetic device 120 are sometimes stored in the auxiliary memory device 140.

As the auxiliary memory device 140, a memory device employing a nonvolatile memory element, such as a flash memory, an MRAM (Magnetoresistive Random Access Memory), a PRAM (Phase change RAM), an ReRAM (Resistive RAM), and an FeRAM (Ferroelectric RAM), or a memory device employing a volatile memory element, such as a DRAM (Dynamic RAM) and an SRAM (Static RAM) may be used, for example. Furthermore, a recording media drive such as a hard disk drive (HDD) and a solid state drive (SSD) may be used, for example.

Alternatively, a memory device that can be detached through the input/output device 150, such as an HDD or an SSD, may be used as the auxiliary memory device 140, for example. Alternatively, a media drive of a computer-readable recording medium such as a flash memory, a Blu-ray disc (registered trademark), a DVD, and a USB memory can be used as the auxiliary memory device 140.

Note that in the case where a memory device located outside the material search system 100 is used as the auxiliary memory device 140, a structure may be employed in which input and output of data to and from with the material search system 100 is performed by wireless communication with the use of the communication device 160.

### [Input/Output Device 150]

The input/output device 150 has a function of controlling input and output of a signal between an external device and the material search system 100. In addition, an HDMI (registered trademark) terminal, a USB terminal, a LAN (Local Area Network) connection terminal, or the like may be used as an external port of the input/output device 150. Furthermore, the input/output device 150 may have a transmission and reception function for optical communication with the use of infrared rays, visible light, ultraviolet rays, or the like. The input/output device 150 also functions as an interface for an information input unit such as a mouse, a keyboard, a pen tablet, or a touch panel (touch sensor).

FIG. 1B shows an example of an XRD profile of a material analyzed. Note that the horizontal axis of the graph shown in FIG. 1B represents 2θ (unit: degree (deg.)) in the case where Cu is used as an X-ray source, and the vertical axis represents intensity (Intensity) in arbitrary unit (a.u.). The XRD profile of a material analyzed is input to the material search system 100 through the input/output device 150.

### [Communication Device 160]

The communication device 160 can perform communication via an antenna. For example, the communication device 160 controls a control signal for connecting the material search system 100 to a computer network in response to instructions from the arithmetic device 120 and transmits the signal to the computer network. Accordingly, communication can be performed by connection of the material search system 100 to a computer network such as the Internet, which is an infrastructure of the World Wide Web (WWW), an intranet, an extranet, a PAN (Personal Area Network), a LAN (Local Area Network), a CAN (Campus Area Network), a MAN (Metropolitan Area Network), a WAN (Wide Area Network), or a GAN (Global Area Network). In the case where a plurality of communication methods are used, a plurality of antennas for the communication methods may be included.

The communication device 160 is provided with a high frequency circuit (RF circuit), for example, to transmit and receive RF signals. The high frequency circuit is a circuit for performing mutual conversion between an electromagnetic signal and an electrical signal in a frequency band that is set by national laws to perform wireless communication with another communication apparatus using the electromagnetic signal. As a practical frequency band, several tens of kilohertz to several tens of gigahertz are generally used. A structure can be employed in which the high frequency circuit connected to an antenna includes a high frequency circuit portion compatible with a plurality of frequency bands and the high frequency circuit portion includes an amplifier, a mixer, a filter, a DSP (Digital Signal Processor), an RF transceiver, or the like. In the case of performing wireless communication, it is possible to use, as a communication protocol or a communication technology, a communication standard such as LTE (Long Term Evolution), GSM (Global System for Mobile Communication: registered trademark), EDGE (Enhanced Data Rates for GSM Evolution), CDMA 2000 (Code Division Multiple Access 2000), or WCDMA (Wideband Code Division Multiple Access: registered trademark), or a communication standard developed by IEEE such as Wi-Fi (registered trademark), Bluetooth (registered trademark), or ZigBee (registered trademark).

The XRD profile of the material to be searched for may be input to the material search system 100 through the communication device 160.

A computer that includes the control device 110, the arithmetic device 120, the memory device 130, the auxiliary memory device 140, and the input/output device 150 or the communication device 160 can function as the material search system 100. For example, when a signal for starting a simulation program according to one embodiment of the present invention is input to the control device 110 through the input/output device 150 or the communication device 160, the control device 110 outputs a signal for reading a program stored in the auxiliary memory device 140 into the memory device 130. By making the memory device 130 read the execution program, the computer can function as the material search system 100. Note that part or all of the program may be stored in the ROM.

In addition, the control device 110 outputs a signal for reading a variety of data, such as setting parameters input through the input/output device 150 or the communication device 160, into the memory device 130. The arithmetic device 120 executes arithmetic processing with use of the program, data, and the like read into the memory device 130. Note that the auxiliary memory device 140 can also be used as the memory device 130. Furthermore, a cache that is provided in the arithmetic device 120 may be used as the memory device 130.

Note that the program for making the computer function as the material search system 100 may be written in a variety of programming languages such as Python (registered trademark), Go, Perl, Ruby, Prolog, Visual Basic (registered trademark), C, C++, Swift, Java (registered trademark), and JavaScript (registered trademark), or a markup language such as html (Hypertext Markup Language) in combination with any of the programming languages. Alternatively, the program may be written in a style sheet language such as CSS (Cascading Style Sheets). Note that a programming language in this specification and the like also includes a markup language and a style sheet language.

The program for making the computer function as the material search system 100 may include a plurality of programs written in the same programming language or a plurality of programs written in different programming languages.

For example, a plurality of programs, each for a different function, may be all written in the same programming language and used collectively as one program. For example, a plurality of programs may be all written in Python and used in combination as one program.

For example, part or all of a plurality of programs, each for a different function, may be written in different programming languages and used in combination as one program. For example, a program written in Python and a program written in JavaScript may be combined and used as one program. For example, a program written in JavaScript may be written in html. Html can be executed using a variety of Web browsers. Thus, the material search system 100 can be achieved with a given computer.

### [Display Device 170]

A variety of display devices can be used as the display device 170. Examples of the display devices include a liquid crystal display device, a light-emitting display device including a light-emitting element such as an EL (Electro Luminescence) element in each pixel, and an electrophoretic display device. In addition, a display device such as a DMD (Digital Micromirror Device), a PDP (Plasma Display Panel), or an FED (Field Emission Display) can be used.

### [Material Database]

The material search system 100 includes a material database 141 that lists physical property values of known materials. The physical property values of the materials may be obtained using CIF (Crystallographic Information File) publicly available from ICSD. Alternatively, PDF (Powder Diffraction File) provided from ICDD (International Centre for Diffraction Data), NIST (National Institute of Standards and Technology), CSD (Cambridge Structural Database), or Pauling File may be used. The physical property values of the materials can also be obtained from a variety of databases other than the above. For the physical property values of the materials, not only data obtained from the databases but also simulation data may be used. Furthermore, the physical property values of the materials may be data obtained through an experiment or the like by the user. Note that the physical property values of the materials are referred to as literature values in some cases.

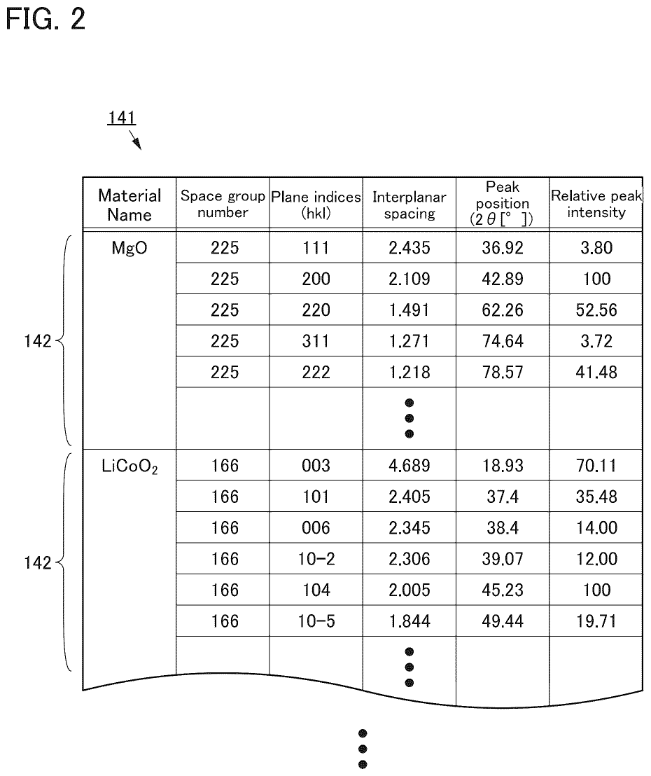

FIG. 2 shows an example of the material database 141 included in the material search system 100. Although information on crystal structures such as lattice constants and space groups of materials is included in the CIF or the like, numerical data on peak positions and relative intensities are not included in some cases. In this case, for each material, the peak positions and relative intensities are calculated in consideration of the wavelength of the X-ray source and the like, and then added to the material database 141 as physical property data 142. Thus, the material database 141 includes the physical property data 142 of a plurality of materials. Note that in the case where a material database that lists the physical property values of materials including the numerical data of the peak positions and relative intensities is used, these values may be added to the material database 141 as they are.

FIG. 2 shows material names, space group numbers, plane indices, interplanar spacings, and peak positions and relative peak intensities when Cu is used as an X-ray source as the items registered in the material database 141. Note that the material database 141 does not necessarily include all of these items and may include other items. A plurality of material databases may be prepared in accordance with the purpose or the like.

Note that the material database 141 may be stored in the auxiliary memory device 140, or may be stored in a server 510, a cloud system 520, or the like that is connected via wired communication or wireless communication through the input/output device 150 or the communication device 160. The material database 141 may be written in the program for making the computer function as the material search system 100. As a specific example, an array of Javascript converted by Python can be embedded in html, necessary data can be extracted from the array with use of Javascript, and a graph and a table can be displayed on the display device 170 with the use of Javascript.

A program for creating the material database 141 can be written in a variety of programming languages such as Python (registered trademark), Go, Perl, Ruby, Prolog, Visual Basic (registered trademark), C, C++, Swift, Java (registered trademark), and JavaScript (registered trademark).

The program for creating the material database 141 may include a plurality of programs written in the same programming language or a plurality of programs written in different programming languages. For example, the material database 141 may be created by adding the physical property data 142 for each material with Python or may be created by adding the physical property data 142 for each material with JavaScript to the material database 141 created with Python.

### [Data Set]

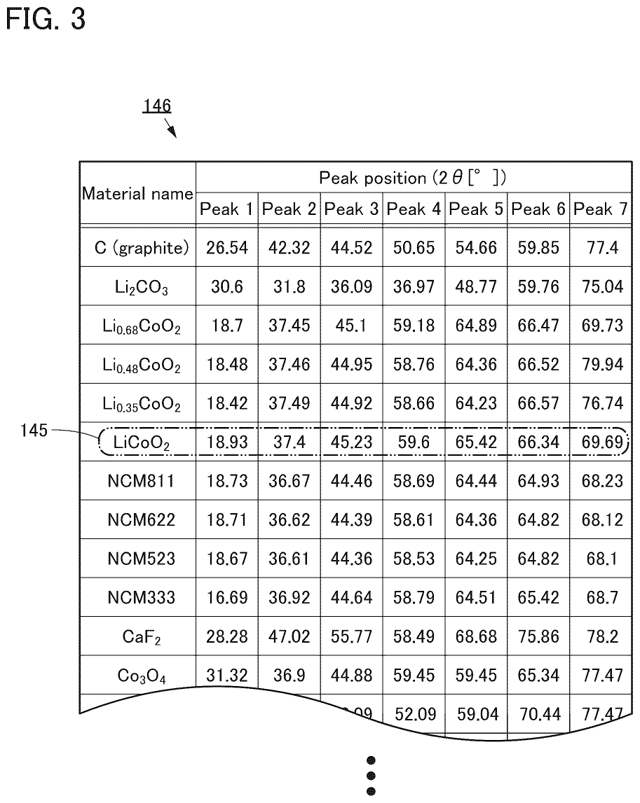

The material search system 100 includes a data set 146 obtained by extracting data necessary for material search from the material database 141. The data set 146 is a collection of a plurality of records 145 generated for each piece of physical property data 142. FIG. 3 shows an example of the data set 146.

The record 145 is generated by extracting R peak positions (R is an integer greater than or equal to 3) in descending order of relative peak intensity for each piece of physical property data 142 included in the material database 141. The data set 146 shown in FIG. 3 is composed of the plurality of records 145 where R is 7. In other words, the data set 146 shown in FIG. 3 is composed of the plurality of records 145 each including information on seven peak positions (a peak 1 to a peak 7) selected in descending order of relative peak intensity.

The data set 146 may include various kinds of information as well as the peak positions. For example, information on plane indices hkl corresponding to the peak positions may be included. By using them, a lattice constant of the estimated material can be calculated.

Note that the number R of the peak positions included in the record 145 is described in detail in the description of the operation example of the material search system 100.

A material matching a sample is searched for using the data set 146. In the case where a constituent element or the like of the sample can be predicted in advance, limitations may be imposed on the records 145 included in the data set 146. For example, in the case where a synthesized material is used for the sample and it is predicted that Li, Co, and O (oxygen) are highly likely to be included in the sample, the records 145 may be limited to those related to cobalt oxide and lithium cobalt oxide-based materials and a material that is expected to be mixed during synthesis of the material and a material containing a constituent element of equipment used in the synthesis of the material.

When the record 145 that is probably not related to the sample is excluded from the data set 146 or is excluded from the search, the search speed and the search accuracy can be increased.

The record 145 included in the data set 146 may be generated by input of peak positions by the user. The record 145 may be generated using information other than that of the material database 141. For example, the peak positions obtained by the theoretical calculation such as simulation may be input. Alternatively, the data set 146 obtained by collecting a predetermined number of peaks in the order of peak intensity from the material database 141 by the user may be used.

Note that for the material names in FIG. 3, Li(Ni_{0.8}Co_{0.1}Mn_{0.1})O₂ is denoted by “NCM811”, and Li(Ni_{0.6}Co_{0.2}Mn_{0.2})O₂ is denoted by “NCM622”. Furthermore, Li(Ni_{0.5}Co_{0.2}Mn_{0.3})O₂ is denoted by “NCM523”, and Li(Ni_{0.33}Co_{0.33}Mn_{0.33})O₂ is denoted by “NCM333”.

A program for creating the data set 146 may be created in the same programming language as the program for creating the material database 141 or may be created in a different programming language. The program for creating the data set 146 may include a plurality of programs written in the same programming language or a plurality of programs written in different programming languages. For example, the material database 141 may be created by adding the physical property data 142 for each material with Python or may be created by adding the record 145 for each material with Javascript to the data set 146 created with Python.

The data set 146 may be written in the program for making the computer function as the material search system 100.

The number of data sets 146 generated in the material search system 100 is not limited to one. For example, a plurality of data sets 146 may be generated in accordance with the purpose or the usage. Alternatively, a frequently used data set 146 among the plurality of data sets 146 may be stored in the memory device 130, and the other data sets 146 may be stored in the auxiliary memory device 140, the server 510, the cloud system 520, or the like.

### <<XRD Analysis Apparatus>>

Here, an XRD analysis apparatus is described. The XRD analysis apparatus and analysis conditions are not particularly limited as long as the apparatus is adjusted appropriately and calibration is performed using a standard sample. The measurement can be performed with the apparatus and conditions as described below, for example.
XRD apparatus: D8 ADVANCE manufactured by Bruker AXS
X-ray source: Cu
Output: 40 kV, 40 mA
Angle of divergence: Div. Slit, 0.5°
Detector: LynxEye
Scanning method: 2θ/θ continuous scanning
Measurement range (2θ): from 15° to 90°
Step width (2θ): 0.01°
Counting time: 1 second/step
Rotation of sample stage: 15 rpm
   As a standard sample used for the adjustment and calibration, a standard sintered alumina plate SRM 1976 from NIST can be used, for example.

In the case where the measurement sample is a powder, the measurement sample can be set by, for example, being put on a glass sample holder or being sprinkled on a reflection-free silicon plate to which grease is applied. In the case where the measurement sample is a positive electrode, the positive electrode is set by being attached to a substrate with a double-sided adhesive tape such that the position of the positive electrode active material layer and the measurement plane required by the apparatus are aligned.

Characteristic X-rays may be monochromatized with the use of a filter or the like or may be monochromatized with XRD data analysis software after an XRD diffraction pattern is obtained. For example, a peak due to CuKα2 radiation can be excluded and only a peak due to CuKal radiation can be extracted by using DEFFRAC.EVA (XRD data analysis software produced by Bruker Corporation). This software can also be used to eliminate the background, for example.

The data subjected to these pretreatments are desirably used as XRD profiles.

### <Operation Example of Material Search System>

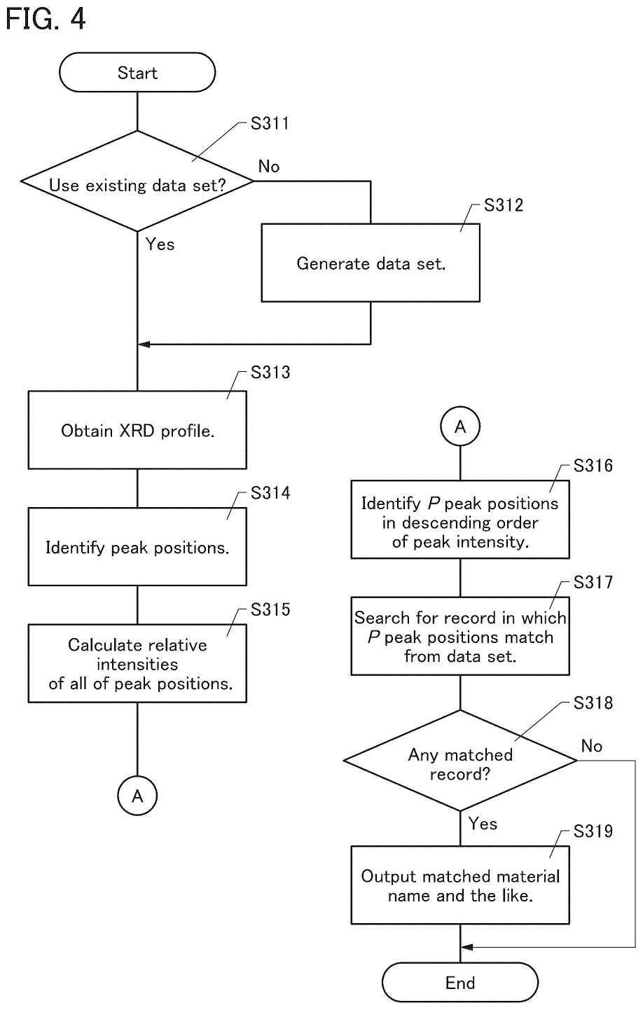

Next, an operation example of the material search system 100 is described as an example of a material search method of one embodiment of the present invention. FIG. 4 is a flow chart for illustrating the operation example of the material search system 100.

First, whether to use an existing data set 146 or a new data set 146 as the data set 146 used in and after Step S317 is determined (Step S311). In the case where the material search system 100 includes a plurality of existing data sets 146, the data set 146 to be used may be determined in accordance with the purpose or the like. In the case where the existing data set 146 is not used, a new data set is generated (Step S312).

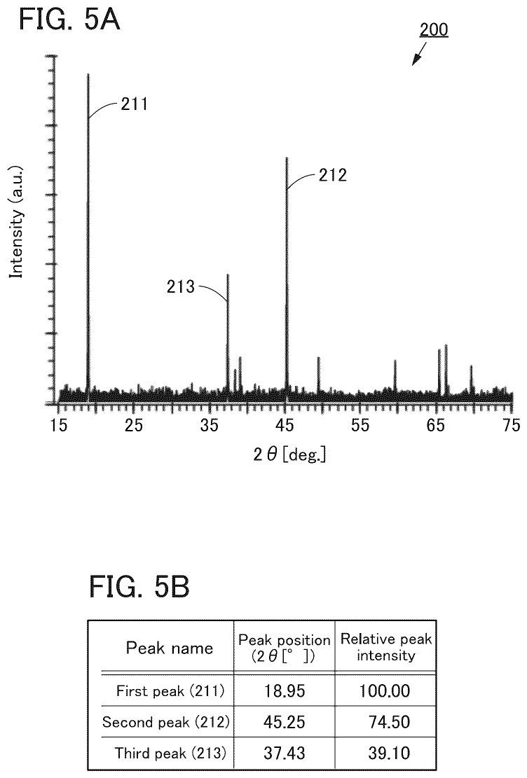

Next, an XRD profile 200 of the sample is obtained (Step S313). The XRD profile 200 may be data input to the material search system 100 through the input/output device 150 or the communication device 160 or data stored in the memory device 130 or the auxiliary memory device 140. Note that a variety of data formats such as an out format, an int format, a csv format, and a dat format are used as the data format of the XRD profile 200. FIG. 5A shows an example of the XRD profile 200. The XRD profile 200 input is stored in the memory device 130, the auxiliary memory device 140, or the like.

Next, peak positions of the obtained XRD profile 200 are identified using the arithmetic device 120 (Step S314). The identification of the peak positions may be performed by differentiation, for example. Data subjected to smoothing or the like may be used as necessary.

Next, peak intensities or relative intensities at all peak positions are calculated with the use of the arithmetic device 120. For example, with the use of a peak having the highest intensity as a reference (e.g., the relative intensity of 100.00), the relative intensities of the other peaks are calculated (Step S315). The calculated peak intensities or relative intensities are stored in the memory device 130, the auxiliary memory device 140, or the like together with the respective peak positions.

Next, P peak positions (P is an integer greater than or equal to 2) are identified in descending order of peak intensity (or relative intensity) (Step S316). In this embodiment, the case where P is 3 is described. That is, the top three peaks having high peak intensities are identified from the XRD profile 200.

FIG. 5A shows a first peak 211, a second peak 212, and a third peak 213 that are identified in descending order of peak intensity. FIG. 5B shows the peak positions and the relative values of the peak intensities of the first peak 211, the second peak 212, and the third peak 213.

The number P of peaks of a sample identified in descending order of peak intensity (or relative intensity) is greater than or equal to 2 and less than or equal to 10, preferably greater than or equal to 2 and less than or equal to 5. As described above, it is difficult to determine a matching or substantially matching material with the use of just one peak among the plurality of peaks appearing in the XRD profile. On the other hand, when the number P of peaks is too large, a peak with a low relative intensity and a noise component are easily confused with each other, whereby the detection accuracy is decreased.

Next, from the data set 146, the record 145 having peaks matching all P peak positions is searched for (Step S317, also referred to as “peak search”). Although the search scope may be the material database 141 instead of the data set 146, the physical property data 142 included in the material database 141 often include information related to the number of peaks exceeding R, whereby the detection accuracy is decreased in some cases. The data set 146 including the plurality of records 145 is generated in advance and the data set 146 is subjected to the search, whereby the presence or absence of a material whose all P peak positions match or substantially match can be efficiently examined. Note that in the case where there is a record 145 in which the number of peaks is less than R, 0 (zero) may be registered as the deficient peak position.

As described above, an error is generated in the intensity of the peak of the sample due to the conditions of the sample, the installation condition of the sample, or the like, and the order of peak intensity is incorrectly recognized in some cases. For example, in the case where the number R of peaks included in the record 145 is less than or equal to the number P of peaks of the sample, any of the P peaks of the sample may be present as or after the R+1-th peak included in the record 145. In this case, the correct record 145 is not detected, whereby the search accuracy is decreased.

Therefore, R needs to be greater than P. That is, R needs to be greater than or equal to P+1. On the other hand, when R is too large, a peak with a low relative intensity and a noise component are easily confused with each other, whereby the detection accuracy is decreased. The number R of peaks included in one record 145 is preferably less than or equal to three times the number P of peaks to be searched for, further preferably less than or equal to six times the number P of peaks to be searched for.

In this embodiment, the case where P is 3 and R is 7 is described. In Step S317, first, whether a peak matching or substantially matching the peak position of the first peak 211 is included in each of the plurality of records 145 included in the data set 146 is examined.

At this time, when the relative difference E (the absolute value of the difference) between the peak positions is within a certain range, it is determined that the peak positions match each other. Specifically, when the relative difference E is less than or equal to 1.00°, preferably less than or equal to 0.50°, further preferably less than or equal to 0.10°, it is determined that the peak positions match each other. Note that the relative difference E is greater than 0 (zero). In this specification and the like, the relative difference E is referred to as “detection range” in some cases. Note that the relative difference E can be set freely by the user.

Next, among the records 145 including the peak matching or substantially matching the peak position of the first peak 211, the records 145 including a peak matching or substantially matching the peak position of the second peak 212 are searched for.

Next, among the records 145 including the peak matching or substantially matching the peak position of each of the first peak 211 and the second peak 212, the record 145 including the peak matching or substantially matching the peak position of the third peak 213 is searched for.

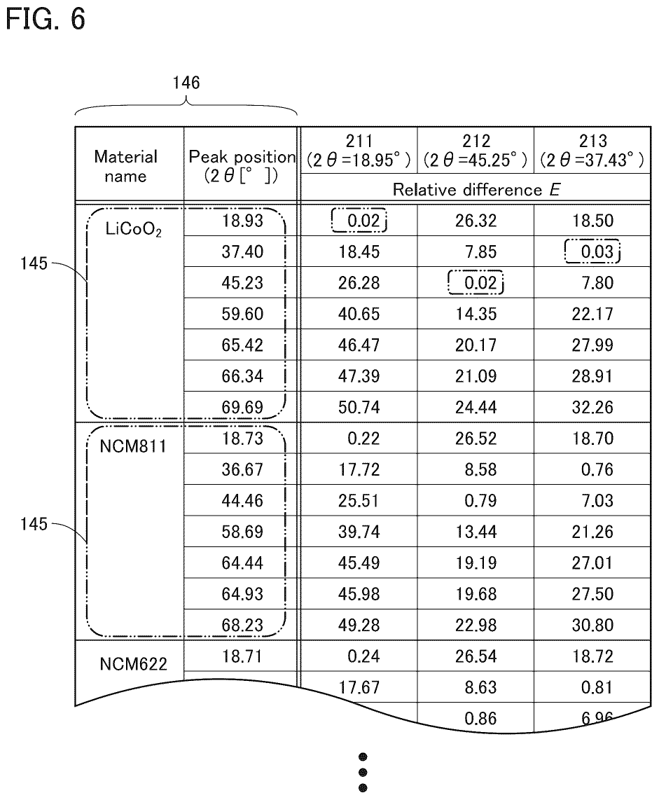

FIG. 6 shows the peak position of each of the first peak 211, the second peak 212, and the third peak 213 of the sample, and the record 145 of LiCoO₂ and the record 145 of NCM811 included in the data set 146. FIG. 6 shows the relative difference E between the peak position of each of the first peak 211, the second peak 212, and the third peak 213 of the sample and each of seven peak positions included in the record 145.

It is found from FIG. 6 that the record 145 generated from the physical property data 142 of LiCoO₂ includes the peak position whose relative difference E from each of the first peak 211, the second peak 212, and the third peak 213 is less than or equal to 0.10°. That is, it can be said that the record 145 of LiCoO₂ has peaks matching or substantially matching the first peak 211, the second peak 212, and the third peak 213.

In the case where the record 145 matching or substantially matching the P peak positions is included in the data set 146 (Step S318), part or the whole of the physical property data 142 related to the record 145, such as the material name, is output to the display device 170, an external device, or the like (Step S319). In this manner, the material constituting the sample can be determined. According to one embodiment of the present invention, a material search using the XRD profile can be achieved without requiring advanced specialized knowledge.

### [Modification Example of Peak Search]

Note that the peak search may be performed as follows. First, a range of the peak position of the first peak 211±the relative difference E (also referred to as “first range”) is set. A range of the peak position of the second peak 212±the relative difference E (also referred to as “second range”) is set. A range of the peak position of the third peak 213±the relative difference E (also referred to as “third range”) is set.

Next, all values of the peak 1 (refer to FIG. 3) to the peak 7 included in the data set 146 are compared with the first range (also referred to as “first comparison processing”), and a truth value of “true” is set for the record 145 having a peak position included in the first range. Next, all values of the peak 1 to the peak 7 included in the data set 146 are compared with the second range (also referred to as “second comparison processing”), and a truth value of “true” is set in the record 145 having a peak position included in the second range. Next, all values of the peak 1 to the peak 7 included in the data set 146 are compared with the third range (also referred to as “third comparison processing”), and a truth value of “true” is set in the record 145 having a peak position included in the third range.

The record 145 in which “true” is set in all of the first comparison processing, the second comparison processing, and the third comparison processing is determined to be the record 145 matching or substantially matching the sample. The first comparison processing, the second comparison processing, and the third comparison processing may be performed sequentially or at the same time.

Note that AI (Artificial Intelligence) processing may be used for the peak search. Machine learning may also be used. For example, a material may be identified with the use of a machine learning algorithm such as a decision tree.

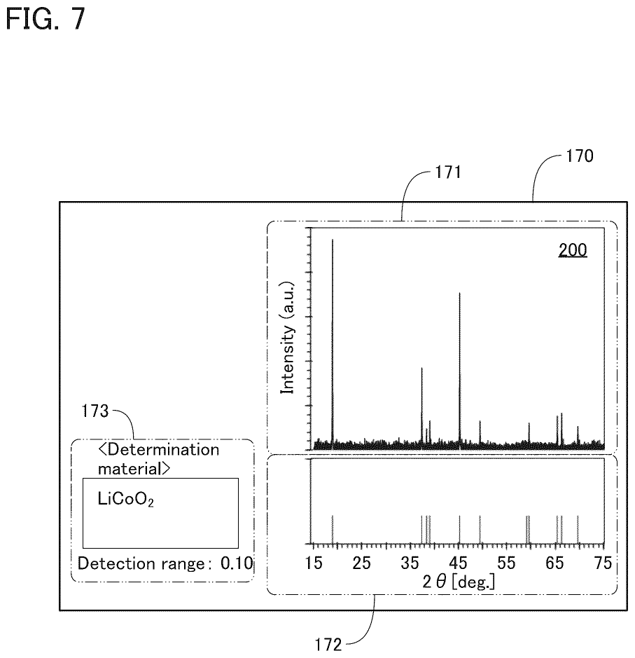

FIG. 7 shows an example of a display screen of the display device 170. The display device 170 includes a first display region 171, a second display region 172, and a third display region 173 on the display screen. The XRD profile of the sample is displayed in the first display region 171. In addition, markers indicating the peak positions calculated from the XRD profile of the material determined to match or substantially match by the search method according to one embodiment of the present invention are shown in the second display region 172.

Note that although the first display region 171 and the second display region 172 are displayed next to each other vertically in FIG. 7, the display regions may be displayed next to each other horizontally, or the content displayed in the second display region 172 may be displayed in the first display region 171. For example, the markers indicating the peak positions of the material that is determined to match or substantially match may be superimposed on the XRD profile of the material. Although the markers are displayed in the second display region 172 as lines perpendicular to the horizontal axis in FIG. 7, the markers do not need to be lines. Note that in the case where the markers are shown by lines perpendicular to the horizontal axis, the lengths of the lines may be associated with relative intensities of the peaks corresponding to the markers or may be displayed with a constant length regardless of the relative intensities of the peaks.

In addition, the horizontal axis (2θ) of the XRD profile displayed in the first display region 171 is preferably aligned with the horizontal axis (2θ) in the second display region 172. Thus, the XRD profile displayed in the first display region 171 and the markers displayed in the second display region 172 can be easily compared with each other.

The third display region 173 displays the name of the material determined to match or substantially match the XRD profile of the sample. At this time, not only the material name but also the physical property value included in the physical property data 142, the management number on the material database 141, and the associated external database management number (e.g., ICSD collection code) may be displayed. Note that information displayed on the display device 170 is not limited to the above. The display device 170 can display a variety of kinds of information. For example, FIG. 7 shows the detection range (the relative difference E) used for the peak search together with the material name.

Furthermore, plane indices at each peak position can be obtained from the physical property data 142 linked to the record 145 which is determined to match or substantially match, and a lattice constant of the sample can be calculated with the use of the plane indices and the peak positions obtained in the measurement of the sample. In the case where the record 145 has information on the plane indices at each peak position, the lattice constant of the sample is calculated with the use of the plane indices of the record 145 and the peak positions obtained in the measurement of the sample. Specifically, the lattice constant of the sample can be calculated using Formula 1.

nλ=2d·sin θ (Formula 1)

In Formula 1, λ is the wavelength of the incident X-ray, d is the interplanar spacing, θ is the incident angle of the X-ray, and n is an integer. With the use of the interplanar spacing, appropriate plane indices, and information on a crystal system of an estimated material, the lattice constants of a variety of crystal systems such as a cubic system, a hexagonal system, and a tetragonal system can be calculated. The calculated lattice constant may be output to the third display region 173 in combination with the material name or the like.

In one embodiment of the present invention, a profile obtained not only by XRD analysis but also by Raman spectroscopy, NMR (Nuclear magnetic resonance) analysis, XPS (X-ray Photoelectron Spectroscopy), or the like can be used for a method used for data analysis. This embodiment can be used in appropriate combination with any of the other embodiments.

### Embodiment 2

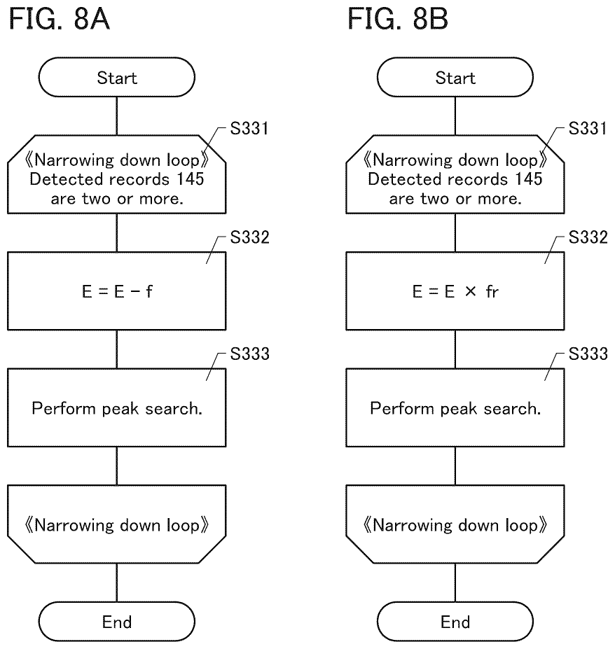

In this embodiment, a method for detecting the record 145 having a peak position that most closely matches the peak position of a sample among a plurality of detected records 145 (also referred to as “narrowing down”) is described as an operation example of the material search system. FIG. 8A and FIG. 8B are flow charts for describing “narrowing down”.

Note that a plurality of material names are displayed on the third display region 173 in some cases. As disclosed in Embodiment 1, in the material search method of one embodiment of the present invention, determination of the matching or substantially matching of peak positions in the peak search is made using the relative difference E (detection range) (Step S317 and Step S318). At this time, when the value of the relative difference E is large, two or more records 145 are detected in some cases. In the case where two or more records 145 are detected as a result of the peak search, “narrowing down” disclosed in this embodiment is performed, so that the record 145 of the material closest to the sample can be detected.

“Narrowing down” can be achieved by repeating the resetting of the relative difference E and the peak search until the detected records 145 are narrowed down to one (also referred to as “narrowing down loop”). In the case where the number of detected records 145 is greater than or equal to 2, the narrowing down loop is started (Step S331).

In the narrowing down loop, first, the relative difference E is reset (Step S332). Specifically, a value obtained by subtracting an adjustment value f from the relative difference E is a new relative difference E (refer to FIG. 8A). The adjustment value f is a value greater than 0 (zero) and less than 1. The adjustment value f is greater than or equal to 0.001° and less than or equal to 0.05°, preferably greater than or equal to 0.005° and less than or equal to 0.02°. For example, the adjustment value f is 0.01°.

Alternatively, the product of the relative difference E and adjustment ratio fr may be a new relative difference E (refer to FIG. 8B). In the case where the product of the relative difference E and the adjustment ratio fr is the new relative difference E, the adjustment ratio fr is greater than or equal to 0.50 and less than 1.00, preferably greater than or equal to 0.80 and less than 1.00. When the product of the relative difference E and the adjustment ratio fr is the new relative difference E, the new relative difference E can be prevented from being a negative value.

Next, a peak search is performed using the relative difference E after the resetting (Step S333). In Step S333, the peak search is performed on the plurality of records 145 detected earlier as a search scope. Note that as in Step S317 described in Embodiment 1, all the records 145 (the data set 146) may be used as the search scope.

The narrowing down loop is repeated until the detected records 145 are narrowed down to one. When the detected records 145 are narrowed down to one, the narrowing down loop is terminated. In this manner, the record 145 having the peak position that most closely matches the peak position of the sample can be detected. That is, the record 145 of the material closest to the sample can be detected.

After that, as in Step S319 described in Embodiment 1, part or the whole of the physical property data 142 related to the record 145, such as the material name, is output to the display device 170, an external device, or the like.

Note that “narrowing down” described in this embodiment may be performed automatically between Step S318 and Step S319 described in Embodiment 1. After the material search method described in Embodiment 1 is executed, “narrowing down” may be performed as needed. For example, “narrowing down” may be executed when the relative difference E is set to 0.50° and the adjustment value f is set to 0.01°.

This embodiment can be used in appropriate combination with any of the other embodiments.

### Embodiment 3

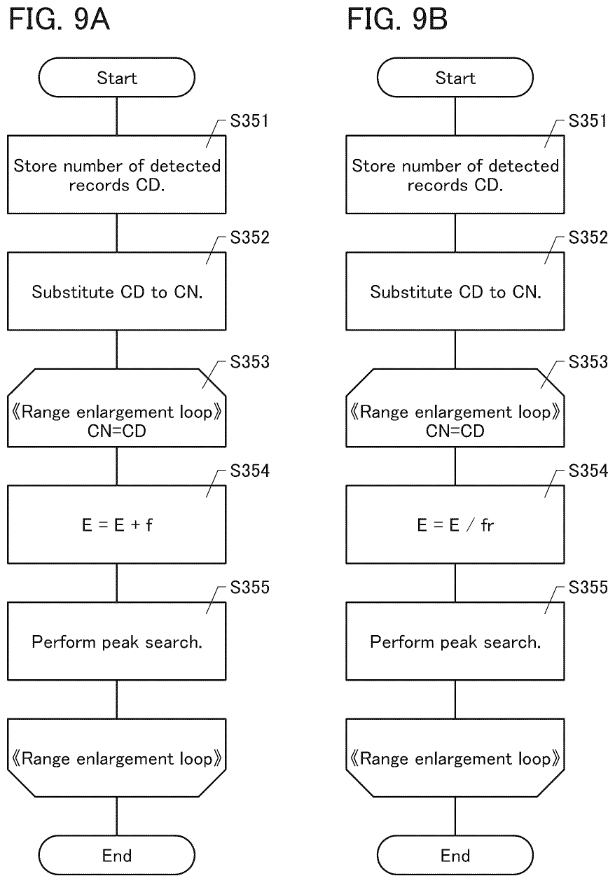

In this embodiment, a method for increasing the number of detected records 145 having peak positions matching the peak positions of a sample by making a detection range (the relative difference E) larger (also referred to as “range enlargement”) is described. FIG. 9A and FIG. 9B are flow charts for describing “range enlargement”.

“Range enlargement” can be achieved by repeating resetting of the relative difference E and peak search (also referred to as “range enlargement loop”). First, the number of previously detected records 145 is stored as the number of detected records CD (Step S351). In the case where no detected record 145 is found, 0 (zero) is stored as the number of detected records CD. Next, the number of detected records CN is substituted to the number of detected records CD, and the number of detected records CN is equal to the number of detected records CD (Step S352).

In the case where the number of detected records CN is equal to the number of detected records CD, the range enlargement loop is started (Step S353).

In the range enlargement loop, first, the relative difference E is reset (Step S354). Specifically, a value obtained by adding the adjustment value f to the relative difference E is a new relative difference E (refer to FIG. 9A). The adjustment value f is a value greater than 0 (zero) and less than 1. The adjustment value f is greater than or equal to 0.001° and less than or equal to 0.05°, preferably greater than or equal to 0.005° and less than or equal to 0.02°. For example, the adjustment value f is 0.01°.

Alternatively, a value obtained by dividing the relative difference E by the adjustment ratio fr may be a new relative difference E (refer to FIG. 9B). In the case where the value obtained by dividing the relative difference E by the adjustment ratio fr is a new relative difference E, the adjustment ratio fr is greater than or equal to 0.70 and less than 1.00, preferably greater than or equal to 0.80 and less than 1.00.

Next, a peak search is performed using the relative difference E after the resetting (Step S355). In Step S355, the peak search is performed with the data set 146 as a search target. The number of detected records 145 in Step S355 is stored as the number of detected records CN.

The range enlargement loop is repeated until the number of detected records CN is larger than the number of detected records CD. When the number of detected records CN is larger than the number of detected records CD, the range enlargement loop is terminated. In this manner, the number of detected records 145 having peak positions matching the peak positions of the sample can be increased.

“Range enlargement” is suitable for the case where the number of detected records CD is 0 (zero) or the case where a material candidate matching the sample is desired to be increased, for example.

This embodiment can be used in appropriate combination with any of the other embodiments.

### Embodiment 4

The material search system 100 of one embodiment of the present invention has a function of extracting materials whose specific peak positions and peak positions in the vicinity thereof are registered in the physical property data 142 from the material database 141 and presenting them to a user (also referred to as “peak filtering”).

With the use of the peak filtering, an impurity material included in the sample, and each of the materials of a sample composed of a plurality of materials and the like can be easily identified. In this embodiment, identification of an impurity material with the use of the peak filtering will be described. Note that in this specification and the like, “impurity material” refers to a material different from a main constituent material, a material unintentionally mixed during sample synthesis, a material unintentionally generated during sample synthesis, or the like.

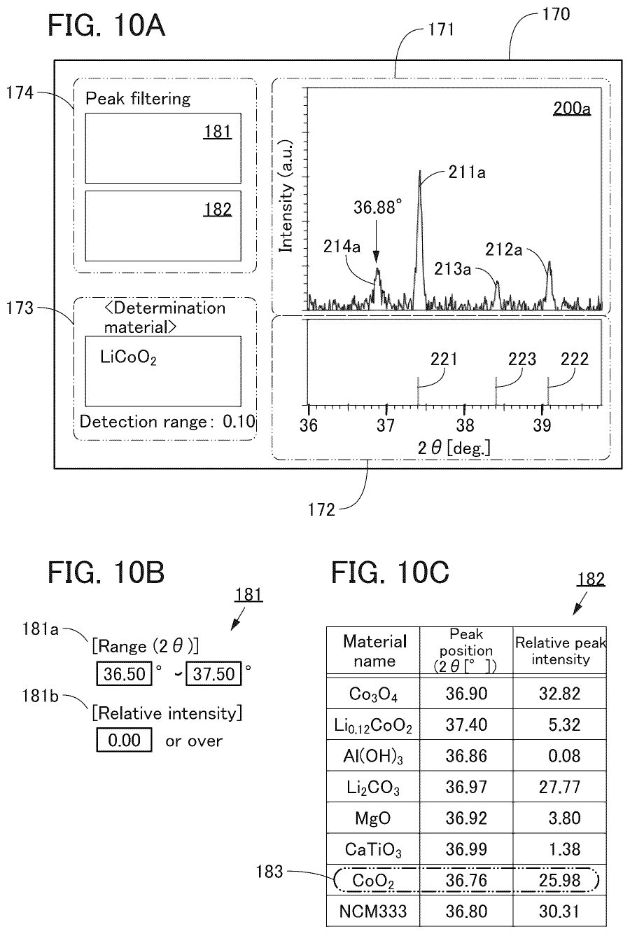

FIG. 10A shows an example of a display screen of the display device 170. The display device 170 shown in FIG. 10A includes the first display region 171, the second display region 172, the third display region 173, and a fourth display region 174 on the display screen.

In FIG. 10A, the first display region 171 shows an XRD profile 200a in which peak positions (2θ) range from 36.00° to 39.70°. In FIG. 10A, a first peak 211a, a second peak 212a, a third peak 213a, and a fourth peak 214a are observed in the XRD profile 200a. In addition, in the second display region 172, markers (a marker 221, a marker 222, and a marker 223) indicating peak positions of the material that is determined to match or substantially match with the use of the search method disclosed in Embodiment 1, and the name of the material is displayed on the third display region 173.

On the fourth display region 174, a filter condition 181 used at the time of performing the peak filtering and a data set 182 extracted from the material database 141 are displayed in accordance with the filter condition 181.

FIG. 10B shows an example of the filter condition 181. In FIG. 10B, a range 181a and a relative intensity 181b of the peak position indicated by 2θ are illustrated as the filter condition 181. The relative intensity 181b is the peak intensity normalized by the maximum peak intensity of the XRD profile 200a. In the peak filtering, a material having peaks in the range 181a and the relative peak intensity is higher than or equal to the relative intensity 181b is extracted from the material database 141, and a set of records 183 each including the extracted material name, the corresponding peak position, and the relative peak intensity is displayed as the data set 182 (refer to FIG. 10C).

Note that the expression “the relative peak intensity is higher than or equal to the relative intensity 181b” means that when the maximum value of the relative intensity is 100, for example, the relative peak intensity is within a range from the relative intensity 181b to 100.

For example, in the XRD profile 200a shown in FIG. 10A, the fourth peak 214a is present at a position of 36.88° other than the first peak 211a, the second peak 212a, and the third peak 213a matching the peak of LiCoO₂. In such a case, it is highly probable that the sample contains the impurity material. By the peak filtering, it is possible to determine which material the fourth peak 214a is derived from.

The determination of materials by the peak filtering is performed in the following manner. First, the range 181a is set. In this embodiment, the peak position of the fourth peak 214a is 36.88°; thus, the range 181a is set to be greater than or equal to 36.50° and less than or equal to 37.50°, for example.

Next, the relative intensity 181b is set. In FIG. 10B, the relative intensity 181b is set to be greater than or equal to 0 (zero). Note that the range 181a and the relative intensity 181b can be set by various means. For example, a setting value may be input to a text box that is set on the screen with the use of a touch panel, a mouse, a keyboard, or the like which is provided to overlap with the display screen of the display device 170. Alternatively, for example, a method may be employed in which a slide bar set on the screen is operated with a mouse, a touch panel, or the like to determine a setting value in accordance with the state of the slide bar. Operation of the slide bar and the operation of setting the setting value in accordance with the state of the slide bar can be achieved by Javascript, for example. The range 181a may be automatically set from the peak position of a target peak of the peak filtering. Note that the slide bar is also referred to as a slider or a slider bar in some cases.

FIG. 10C shows an example of the data set 182 generated from the material database 141 in accordance with the filter condition 181. The data set 182 shown in FIG. 10C indicates that a plurality of materials having peaks matching the filter condition 181 are found. Among these peaks, a peak with an extremely low relative intensity is difficult to distinguish from a noise component of an XRD profile. When the data set 182 includes a peak with an extremely low relative intensity, it takes time for accurate determination and the determination efficiency decreases. Information on a peak that is difficult to distinguish from the noise component is one factor of a reduction in determination accuracy and thus is preferably excluded from the data set 182.

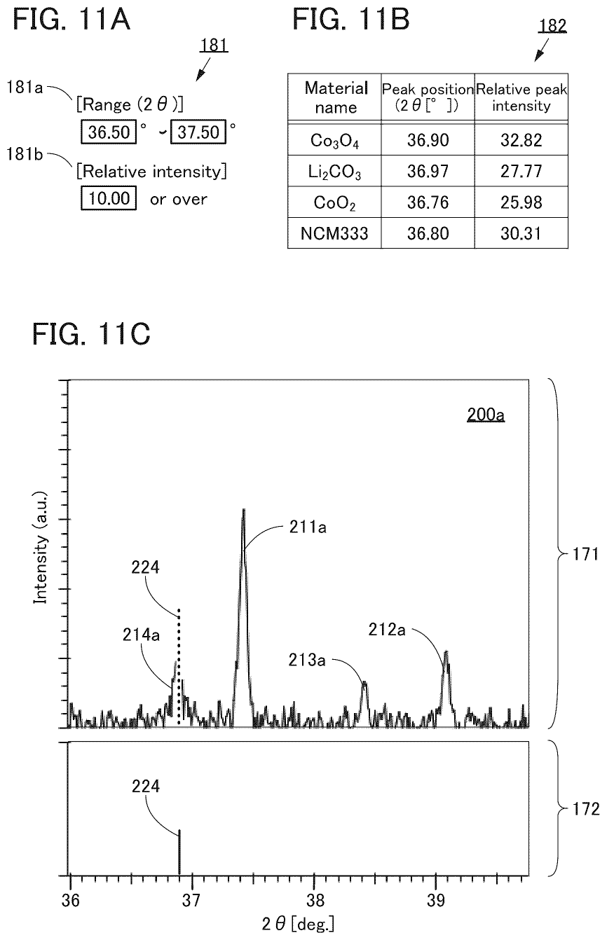

The material having a peak with an extremely low relative intensity extracted under the condition of the filter condition 181 can be excluded by increasing the relative intensity 181b of the filter condition 181. For example, when the relative intensity 181b is increased (refer to FIG. 11A), the data set 182 in which the material having a peak with an extremely low relative intensity is excluded is obtained (refer to FIG. 11B). Note that the setting value of the relative intensity 181b for excluding the noise component from the data set 182 is preferably greater than or equal to 5 and less than or equal to 15. Meanwhile, in the case where the material has a crystal structure (e.g., Li₂CO₃) having a large number of peaks of 10 or more in the measurement range or the like, the setting value may be set to be less than or equal to 30 or less than or equal to 60. The setting value of the relative intensity 181b for excluding the noise component is set as appropriate.

It is found from the data set 182 shown in FIG. 11B that a peak at a peak position of 36.90° of Co₃O₄ is closest to the fourth peak 214a, and the relative intensity exceeding the relative intensity 181b is obtained. Thus, it can be determined that the fourth peak 214a is highly likely to be derived from Co₃O₄. That is, it can be determined that Co₃O₄ is highly likely to be contained in the sample.

The peaks of materials included in the data set 182 is preferably superimposed on the XRD profile 200a displayed on the first display region 171. For example, a peak of a given material is selected from the data set 182, and a line is displayed at a position matching the peak on the XRD profile 200a.

FIG. 11C is a graph showing a display example of the first display region 171 and the second display region 172. In FIG. 11C, in the first display region 171, the marker 224 indicating the position at the peak position 36.90° derived from Co₃O₄ is superimposed on the XRD profile 200a. Note that the marker 224 may be shown in the second display region 172 or both of the first display region 171 and the second display region 172.

In FIG. 11C, the marker 224 shown in the first display region 171 is indicated by a dashed line perpendicular to the horizontal axis, and the marker 224 shown in the second display region 172 is indicated by a straight line perpendicular to the horizontal axis. Note that markers indicating peak positions do not need to be lines. Note that in the case where markers are indicated by lines perpendicular to the horizontal axis, the length of the lines may be associated with the relative peak intensities corresponding to the markers or may be displayed with a constant length regardless of the relative peak intensities.

A material in the data set 182 displayed on the display screen is selected freely or sequentially, and the markers indicating peak positions of the material are superimposed on the XRD profile 200a, whereby a peak closest to the fourth peak 214a can be visually captured.

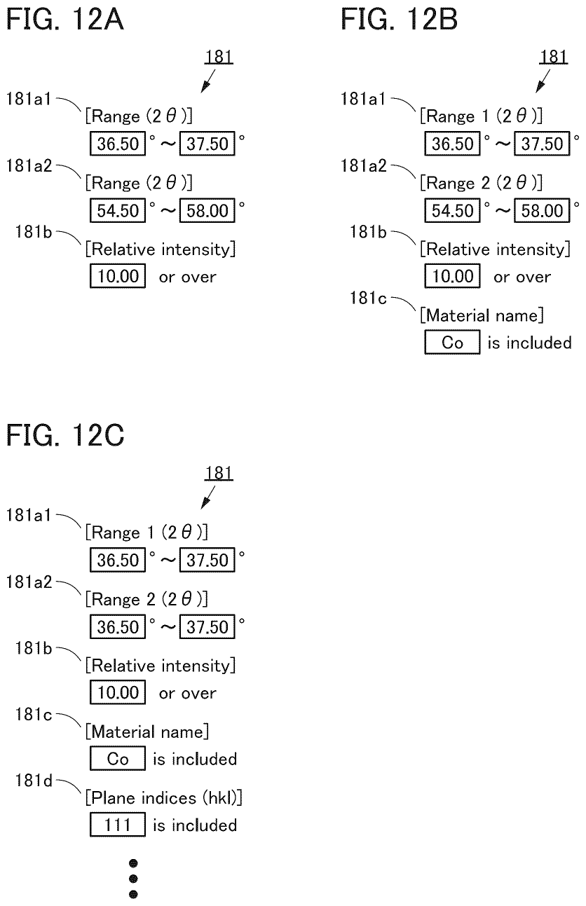

The number of the ranges 181a to be set as the filter condition 181 is not limited to one. FIG. 12A shows an example in which two peak ranges, which are a range 181a1 and a range 181a2, are set as the filter condition 181. When the two ranges 181a are set, both of a material having a peak in the range 181a1 and a material having a peak in the range 181a2 can be extracted from the material database 141. Thus, a material in which each of a plurality of peaks is derived from can be efficiently identified. Note that three or more ranges 181a may be set as the filter condition 181.

The conditions for setting the filter condition 181 are not limited to the range 181a and the relative intensity 181b. For example, a material-name-including character 181c may be set as the filter condition 181 (FIG. 12B). For example, plane indices 181d may be set as the filter condition 181 (FIG. 12C). Other than the above, a variety of conditions may be set for setting the filter condition 181.

This embodiment can be used in appropriate combination with any of the other embodiments.

### REFERENCE NUMERALS

100: material search system, 110: control device, 120: arithmetic device, 130: memory device, 140: auxiliary memory device, 141: material database, 142: physical property data, 145: record, 146: data set, 150: input/output device, 160: communication device, 170: display device, 171: first display region, 172: second display region, 173: third display region, 200: XRD profile, 211: first peak, 212: second peak, 213: third peak, 510: server, 520: cloud system

1. A material search method using an XRD profile and a first data set,
wherein the first data set comprises a plurality of records each comprising R first peak positions extracted from each of a plurality of pieces of physical property data of known materials in descending order of a peak intensity,
wherein the material search method comprises:
identifying a plurality of second peak positions and intensities from the XRD profile of a first material;
obtaining P second peak positions in descending order of a peak intensity from the plurality of second peak positions and intensities;
searching the first data set for the record comprising the first peak positions matching the P second peak positions; and
determining the first material to be the same as the known material related to the record in the case where the matched record is found,
wherein P is an integer greater than or equal to 2 and less than or equal to 10, and
wherein R is an integer greater than P.

2. The material search method according to claim 1,
wherein R is less than or equal to 6 times P.

3. The material search method according to claim 1,
wherein the record comprises R plane indices corresponding to the R first peak positions, and
wherein the material search method further comprises calculating a lattice constant of the first material determined to be the same as the known material using the first peak positions and the plane indices.

4. A program for executing the material search method according to claim 1 on a computer.

5. A computer-readable recording medium comprising the program according to claim 4.

6. The program according to claim 4, wherein the program is written in html.

7. A material search system using an XRD profile and a first data set,
wherein the first data set comprises a plurality of records each comprising R first peak positions extracted from each of a plurality of pieces of physical property data of known materials in descending order of a peak intensity,
wherein the material search system is configured to identify a plurality of second peak positions and intensities from the XRD profile of a first material,
wherein the material search system is configured to obtain P second peak positions in descending order of a peak intensity from the plurality of second peak positions and intensities,
wherein the material search system is configure to search the first data set for the first peak positions matching the P second peak positions,
wherein the material search system is configured to determine the first material to be the same as the known material related to the record in the case where the matched record is found,
wherein P is an integer greater than or equal to 2 and less than or equal to 10, and wherein R is an integer greater than P.

8. The material search system according to claim 7,
wherein R is less than or equal to 6 times P.

9. The material search system according to claim 7,
wherein the record comprises R plane indices corresponding to the R first peak positions, and
wherein the material search system is configured to calculate a lattice constant of the first material determined to be the same as the known material using the first peak positions and the plane indices.

10. A program for implementing the material search system according to claim 1 on a computer.

11. A computer-readable recording medium comprising the program according to claim 10.

12. The program according to claim 10, wherein the program is written in html.

13. A material search system using an XRD profile and a second data set,
wherein the second data set comprises a plurality of records,
wherein the record comprises a name of a material having a peak at a peak position in a specified range and the peak position,
wherein the material search system is configured to display the XRD profile on a display device,
wherein the material search system is configured to display the second data set on the display device, and
wherein the material search system has a function of displaying is configured to display, on the display device, a marker indicating the peak position of the record selected from the second data set.

14. The material search system according to claim 13,
wherein the peak has a relative intensity in the specified range.

15. The material search system according to claim 13,
wherein the material search system is configured to display, on the display device, the marker and the XRD profile overlapping with each other.

16. A program for implementing the material search system according to claim 13 on a computer.

17. A computer-readable recording medium comprising the program according to claim 16.

18. The program according to claim 16, wherein the program is written in html.

19. A program for implementing the material search system according to claim 15 on a computer.

20. A computer-readable recording medium comprising the program according to claim 19.

21. The program according to claim 19, wherein the program is written in html.
